(19) United States
(12) Patent Application Publication
(10) Pub. No.: US 2023/0391732 A1
(43) Pub. Date: Dec. 7, 2023

(54) MULTI-EFFICACY PYRAZINE COMPOUND, PREPARATION METHOD AND USE THEREOF

(71) Applicant: SHENZHEN OLIVE BIOPHARMACEUTICALS CO., LTD, Shenzhen (CN)

(72) Inventors: Yongmei XIE, Shenzhen (CN); Shupeng LI, Shenzhen (CN); Xifei YANG, Shenzhen (CN)

(73) Assignee: SHENZHEN OLIVE BIOPHARMACEUTICALS CO., LTD, Shenzhen (CN)

(21) Appl. No.: 18/018,885

(22) PCT Filed: Jul. 30, 2021
(86) PCT No.: PCT/CN2021/109563

§ 371 Date: Aug. 25, 2023

(30) Foreign Application Priority Data
Jul. 31, 2020 (CN) 202010759391.0
Jul. 31, 2020 (CN) 202010759395.9

(51) Int. Cl. C07D 241/24; C07F 7/18; A61P 25/28; A61P 25/16; A61P 3/10

(52) U.S. Cl. CPC C07D 241/24; C07F 7/18; A61P 25/28; A61P 25/16; A61P 3/10

(57) ABSTRACT

The present disclosure provides a pyrazine compound, a stereoisomer, and a tautomer, and a pharmaceutically acceptable salt thereof in treating a neurodegenerative disease (ND) including Alzheimer's disease, Parkinson's disease, Huntington's disease, frontotemporal dementia (FTD), vascular dementia, HIV-related dementia, multiple sclerosis, progressive lateral sclerosis, Friedreich's ataxia, neuropathic pain, or glaucoma, diabetes mellitus (DM) and a DM-related complication, an inflammation, an oxidative damage, and a mitochondrial disorder-related disease.

### CROSS REFERENCE TO RELATED APPLICATIONS

The present application is a national stage application of International Patent Application No. PCT/CN2021/109563, filed on Jul. 30, 2021, which claims the benefit and priority of Chinese Patent Application No. CN202010759395.9 entitled “MULTI-EFFICACY PYRAZINE COMPOUND AND PREPARATION METHOD THEREOF”, and Application No. CN202010759391.0 entitled “USE OF MULTI-EFFICACY PYRAZINE COMPOUND IN PREPARATION OF DRUG”, filed on Jul. 31, 2020, all of which are incorporated herein by reference in their entities.

### TECHNICAL FIELD

The present disclosure relates to the technical field of medicines, in particular to a multi-efficacy pyrazine compound, a preparation method and use thereof.

### BACKGROUND ART

Neurodegenerative disease (ND) is a chronic disease that leads to the progressive death of neurons, including Alzheimer's disease, Parkinson's disease, and Huntington's disease. The ND generally brings huge pain to patients and heavy burden to their families. As the population aging aggravates, it is expected that, the ND will replace cancer as the second largest group of diseases that cause human death by 2040. However, there is currently no drug in the world that can effectively treat the ND.

The pathology of ND is closely related to oxidative stress, mitochondrial dysfunction, Ca²⁺ influx, immune inflammation, autophagy and metal ions, such that the ND is a complex disease with multiple etiological factors. The traditional single-target and high-selectivity drug development strategy is not effective in the development of new drugs for the ND. Traditional Chinese medicine has become a research hotspot of anti-ND drugs in recent years due to multiple therapeutic targets, small toxic and side effects, and desirable synergistic effect.

Diabetes mellitus (DM), as a lifelong metabolic disease caused by insulin secretion defect or insulin utilization disorder, is mainly characterized by hyperglycemia. With the improvement of residents' living standards and the changes in dietary structure, the incidence of DM is increasing year by year and the age of onset is becoming younger and younger. Diabetic nephropathy (DN) is one of the common chronic complications of the DM, and the incidence rate is about 20% to 40% in the diabetic population, about 50% of DN patients may die of terminal renal failure in a later stage, which is the main cause of death from chronic kidney diseases. The DN has the characteristics of extremely hidden, complex and diverse pathogenesis, and there is still a lack of effective treatment in clinic.

Through long-term researches, a pyrazine compound is found in the present disclosure, which has a therapeutic effect on the ND and DM.

### SUMMARY

The present disclosure provides a pyrazine compound, a stereoisomer, and a tautomer, and a pharmaceutically acceptable salt thereof, where the pyrazine compound is shown in formula I:

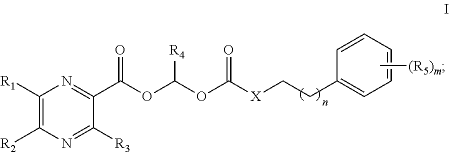

in formula I, wherein, X is selected from the group consisting of O, S, Se, and NR₆; R₁, R₂, R₃, R₄, R₅, and R₆ each are independently selected from the group consisting of H, deuterium, halogen, hydroxyl, amino, carboxyl, acylamino, ester, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkoxy, substituted or unsubstituted alkylcarboxyl, substituted or unsubstituted alkylester, substituted or unsubstituted -alkyl-OH, substituted or unsubstituted alkoxy, substituted or unsubstituted alkylamino, substituted or unsubstituted -alkyl-NH₂, substituted or unsubstituted aryl, substituted or unsubstituted heterocyclic aryl, substituted or unsubstituted carbonate, substituted or unsubstituted carbamate, substituted or unsubstituted -alkyl-acylamino, substituted or unsubstituted -aminoalkylcarboxylate, and deuterated derivatives of the above groups; and n is 0 to 6, m is 0 to 5.

In some embodiments, n may be 0, 1, 2, 3, 4, 5, or 6; and m may be 0, 1, 2, 3, 4, or 5.

Further, R₁, R₂, and R₃ each may be independently selected from the group consisting of methyl and deuterated methyl, X may be selected from the group consisting of O, S, Se, and NR₆; and R₄ may be selected from the group consisting of H and C₁-C₆ alkyl.

Further, n may be 1, X may be selected from the group consisting of O, S, Se, and NH; and R₄ may be selected from the group consisting of H and C₁-C₆ alkyl.

Further, X may be O, n may be 1; and R₄ may be selected from the group consisting of H and C₁-C₆ alkyl.

Further, the compound may be shown as follows:

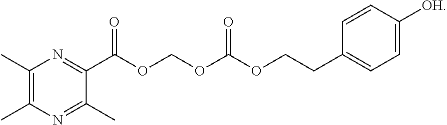 [SMILES: Cc1nc(C)c(C(=O)OCOC(=O)OCCc2ccc(O)cc2)nc1C]

Further, the pharmaceutically acceptable salt of the pyrazine compound may be a salt obtained by reaction of the compound with hydrochloric acid, sulfuric acid, phosphoric acid, hydrobromic acid, nitric acid, salicylic acid, oxalic acid, benzoic acid, maleic acid, fumaric acid, citric acid, succinic acid, tartaric acid, C₁₋₆ fatty carboxylic acid, C₁₋₆ alkyl sulfonic acid, benzenesulfonic acid, p-toluenesulfonic acid, or camphorsulfonic acid.

The present disclosure also provides a compound, having a structural formula as follows:

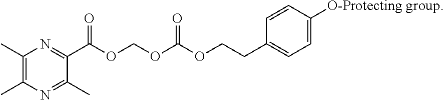

The present disclosure also provides a compound, having a structural formula as follows:

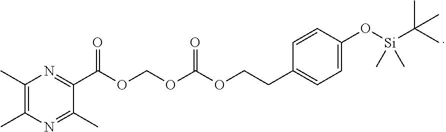 [SMILES: Cc1nc(C)c(C(=O)OCOC(=O)OCCc2ccc(O[Si](C)(C)C(C)(C)C)cc2)nc1C]

The present disclosure also provides a preparation method of a compound, including the following steps:

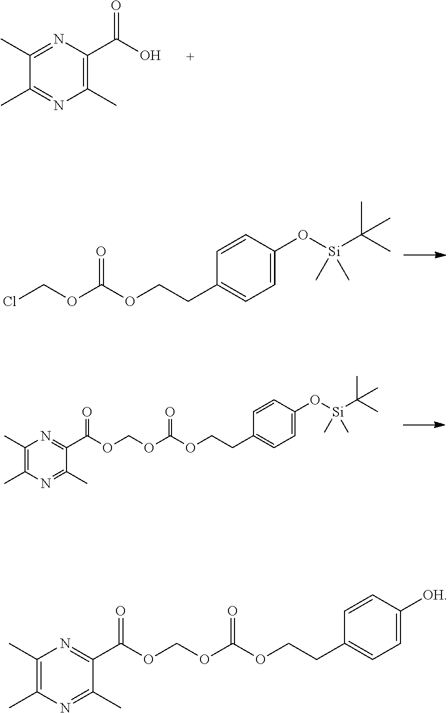

The present disclosure also provides a preparation method of a compound, including the following steps:

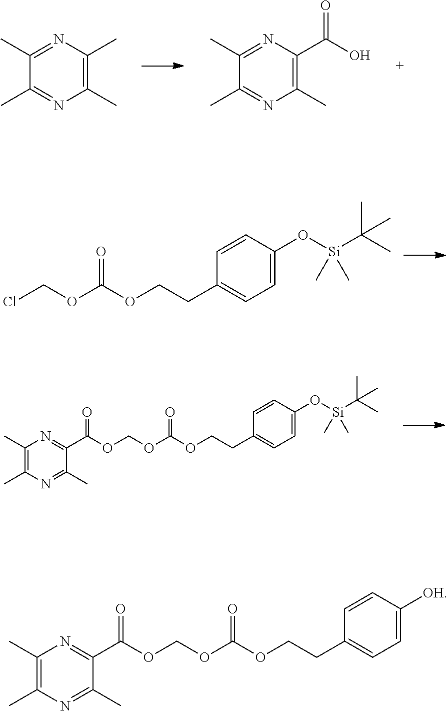

The present disclosure also provides a preparation method of a compound, including the following steps:

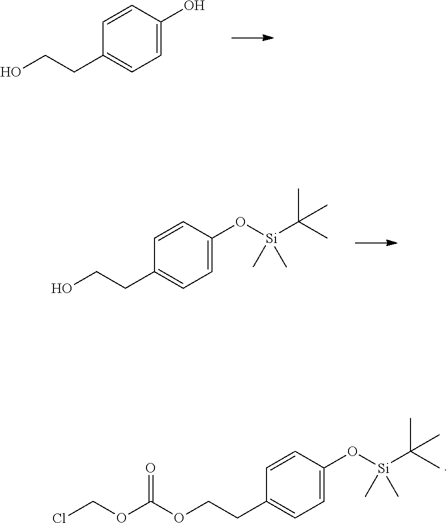 [SMILES: OCCc1ccc(O)cc1 ; CC(C)(C)[Si](C)(C)Oc1ccc(CCO)cc1 ; CC(C)(C)[Si](C)(C)Oc1ccc(CCOC(=O)OCCl)cc1]

The present disclosure also provides a pharmaceutical composition, including a therapeutically effective amount of one or more of the pyrazine compound, the stereoisomer, and the tautomer, and the pharmaceutically acceptable salt thereof as described above.

Use of the pyrazine compound, the stereoisomer, and the tautomer, and the pharmaceutically acceptable salt thereof as described above in treating an ND including Alzheimer's disease, Parkinson's disease, Huntington's disease, frontotemporal dementia (FTD), vascular dementia, HIV-related dementia, multiple sclerosis, progressive lateral sclerosis, neuropathic pain or glaucoma, DM and a DM-related complication, an inflammation, an oxidative damage, and a mitochondrial disease.

The present disclosure also provides a pharmaceutical composition, including a therapeutically effective amount of one or more of the pyrazine compound, the stereoisomer, and the tautomer, and the pharmaceutically acceptable salt thereof as described above.

The present disclosure also provides use of the pyrazine compound, the stereoisomer, and the tautomer, and the pharmaceutically acceptable salt thereof in treating an ND including Alzheimer's disease, Parkinson's disease, Huntington's disease, frontotemporal dementia (FTD), vascular dementia, HIV-related dementia, multiple sclerosis, progressive lateral sclerosis, neuropathic pain or glaucoma, DM and a DM-related complication, an inflammation, an oxidative damage, and a mitochondrial disease.

The pyrazine compound provided by the present disclosure improves glucose and lipid metabolism, reduces urinary protein, and has a neuroprotective activity, can resist inflammation, improve memory damage and resist oxidative damage, and has a therapeutic effect on amyotrophic lateral sclerosis (ALS), and can prevent and/or treat Parkinson's disease and Alzheimer's disease.

The present disclosure also provides a pharmaceutical composition, including a therapeutically effective amount of any one or more of the pyrazine compound, the stereoisomer, and the tautomer, and the pharmaceutically acceptable salt thereof as described above.

In some embodiments, the pharmaceutical composition may further include one or more pharmaceutically acceptable carriers or excipients.

In some embodiments, the pharmaceutical composition may further include other therapeutic agents.

In one embodiment of the present disclosure, the compound used may be administered by oral, injection, subcutaneous, respiratory, transdermal, parenteral, rectal, topical, intravenous, intramuscular, or by other means in a dose unit formulation comprising a conventional pharmaceutically acceptable carrier. Further, the pharmaceutical composition may be formulated into any pharmaceutical form, such as a tablet, a granule, an injection, a gel, a pill, a capsule, a suppository, an implant, a nano preparation, and a powder for injection. Some dosage forms such as the tablet and the capsule may be subdivided into appropriate dosage unit forms containing an appropriate amount of an active component, such as an effective amount to achieve a desired purpose.

The carrier includes excipients and diluents, and must be of sufficiently high purity and sufficiently low toxicity to be suitable for administration to patients to be treated. The carrier may be inert or if may itself have a pharmaceutical benefit.

The carrier may include, but is not limited to: a diluent such as a filler, and a bulking agent, a binder, a lubricant, an anti-caking agent, a disintegrant, a sweetener, a buffer, a preservative, a solubilizer, an isotonic agent, a suspending agent and a dispersing agent, a wetting agent or an emulsifying agent, a flavoring agent and a perfuming agent, a thickening agent and a vehicle. The exemplary pharmaceutically acceptable carrier may include sugar, starch, cellulose, malt, gelatin, talc, and vegetable oil. An optional activator may be included in the pharmaceutical composition, which do not substantially affect the activity of the compound of the present disclosure.

### Terminology

The term “stereoisomer” or “optical isomer” refers to a compound that has a same chemical composition but differ in arrangement of atoms or groups in space, which includes a “diastereomer” and an “enantiomer”.

The term “diastereomer” refers to a stereoisomer that has two or more chiral centers, and whose molecules are not mirror images of each other. The diastereomer has different physical properties such as melting point, boiling point, spectral properties and reactivity. A mixture of the diastereomers can be separated under high-resolution analytical steps such as electrophoresis and crystallization, using, for example, a chiral HPLC column in the presence of a resolving agent or chromatography.

The term “enantiomer” refers to two stereoisomers of a compound that are non-superimposable mirror images of each other. A 50:50 mixture of the enantiomers is referred to as a racemic mixture or a racemate, which can occur during a chemical reaction or process where no stereoselectivity or stereospecificity is available.

The term “alkyl” includes both branched and straight-chain saturated aliphatic hydrocarbon groups and has a specified number of carbon atoms, generally from 1 to about 12 carbon atoms. For example, the term C₁-C₆ alkyl as used herein refers to an alkyl having 1 to about 6 carbon atoms. When C₀-Cₙ alkyl is used herein in conjunction with another group, (phenyl)C₀-C₄ alkyl is taken as an example to describe a designated group. In this case, the phenyl is directly bonded by a single covalent bond (C₀) or connected by an alkyl chain having a specified number of carbon atoms (in this case, 1 to about 4 carbon atoms). The alkyl includes, but is not limited to: methyl, ethyl, n-propyl, isopropyl, n-butyl, 3-methylbutyl, tert-butyl, n-pentyl, or sec-pentyl.

The term “alkenyl” or “alkenyl” refers to straight and branched hydrocarbon chains including one or more unsaturated carbon-carbon bonds that may occur at any stable point along the chain. As used herein, the alkenyl generally has from 2 to about 12 carbon atoms. Preferably, the alkenyl is a lower alkenyl having from 2 to about 8 carbon atoms, such as: C₂-C₈, C₂-C₆, or C₂-C₄ alkenyl. Examples of alkenyl include vinyl, propenyl, or butenyl.

The term “cycloalkyl” preferably refers to a cyclic alkyl with a monocyclic, bicyclic, tricyclic, bridged-cyclic, and spirocyclic structure and having 3 to 15 carbon atoms, preferably including cyclopropane, cyclopentane, and cyclohexane.

The term “alkoxy” refers to an alkyl as defined above, having a specified number of carbon atoms connected by an oxygen bridge. Examples of alkoxy include, but are not limited to: methoxy, ethoxy, 3-hexyloxy or 3-methylpentyloxy.

The term “heterocycle” refers to a 5- to 8-membered saturated ring, a partially unsaturated ring, or an aromatic ring containing 1 to about 4 heteroatoms selected from N, O, and S and using carbon as the remaining ring atoms. The heterocycle can also refer to a 7- to 11-membered saturated, partially unsaturated, or aromatic heterocycle system; and a 10- to 15-membered tricyclic system; the system contains at least 1 heteroatom selected from a polycyclic system of N, O and S and up to about 4 heteroatoms independently selected from N, O and S in each ring of the polycyclic system. Unless otherwise specified, a heterocycle can be connected to a group where the heterocycle is substituted at any heteroatom and carbon atom and results in a stable structure. When specified, the heterocycle herein may be substituted on carbon atom or nitrogen atom so long as the resulting compound is stable. Optionally, nitrogen atoms in the heterocycle can be quaternized. Preferably, the total number of heteroatoms in heterocyclyl is not more than 4 and preferably the total number of S and O atoms in heterocyclyl is not more than 2, more preferably not more than 1. Examples of heterocyclyl include: pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, dihydroisoindolyl, 5,6,7,8-tetrahydroisoquinoline, pyridyl, pyrimidinyl, furanyl, thienyl, pyrrolyl, pyrazolyl, pyrrolidinyl, morpholinyl, piperazinyl, piperidinyl, or pyrrolidinyl.

The term “aryl” or “heteroaryl” refers to a stable 5- or 6-membered monocyclic ring or polycyclic ring containing 1 to 4, or preferably 1 to 3 heteroatoms selected from N, O and S and using carbon as the remaining ring atoms. When the total number of S and O atoms in heteroaryl exceeds 1, these heteroatoms are not adjacent to each other. Preferably, the total number of S and O atoms in heteroaryl is not more than 2. Especially preferably, the total number of S and O atoms in heteroaryl is not more than 1. Optionally, nitrogen atoms in the heterocycle can be quaternized. When specified, these heteroaryl may also be substituted with carbon or non-carbon atoms or groups. Such substitution may include fusing with a 5- to 7-membered saturated cyclic group optionally containing 1 or 2 heteroatoms independently selected from N, O, and S to form, for example, [1,3]dioxazolo[4,5-c]pyridyl. Examples of heteroaryl include, but are not limited to: pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolyl, pyrrolyl, pyrazolyl, benzo[b]phenylthio, isoquinolinyl, quinazolinyl, quinoxalinyl, thienyl, isoindolyl, or 5,6,7,8-tetrahydroisoquinoline.

The term “pharmaceutically acceptable salt” or “salt of compound” are derivatives of the disclosed compounds, wherein the parent compound is modified by preparing a non-toxic acid or base addition salts thereof; the two terms also refer to a pharmaceutically acceptable solvate, including hydrates, of these compounds and these salts. Examples of pharmaceutically acceptable salt include, but are not limited to: inorganic or organic acid addition salts of basic residues such as amines, base or organic addition salts of acidic residues such as carboxylic acid, and combinations including one or more of the above salts. The pharmaceutically acceptable salt includes nontoxic and quaternary ammonium salts such as a parent compound formed from non-toxic inorganic or organic acids. For example, the non-toxic acid salt includes those derived from inorganic acids such as: hydrochloric acid, hydrobromic acid, sulfuric acid, sulfamic acid, phosphoric acid, nitric acid, other acceptable inorganic salt includes metal salts such as: sodium salts, potassium salts and cesium salts; alkaline earth metal salt such as: calcium salts and magnesium salts; and combinations include one or more of the above salts.

An organic salt of the compounds includes those prepared from organic acids such as acetic acid, trifluoroacetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, methanesulfonic acid, ethanesulfonic acid, benzenesulfonic acid, p-aminobenzenesulfonic acid, 2-acetoxybenzoic acid, fumaric acid, p-toluenesulfonic acid, methanesulfonic acid, ethanedisulfonic acid, oxalic acid, isethionic acid, HOOC—(CH₂)n-COOH (where n is 0 to 4); organic amine salts, such as: triethylamine salts, pyridine salts, picoline salts, ethanolamine salts, triethanolamine salts, dicyclohexylamine salts, and N,N′-dibenzylethylenediamine salts, and amino acid salts, such as: arginine, aspartate, and glutamate; and combinations including one or more of the above salts.

### BRIEF DESCRIPTION OF THE DRAWINGS

FIG. 1 shows that OLB-3 significantly reduce death of SH-SY5Y cells caused by OGD;

FIG. 2 shows that OLB-3 significantly reduce a urinary protein level of db/db mice;

FIG. 3 shows that OLB-3 significantly improve memory impairment in the 5*FAD mice;

FIG. 4 shows that OLB-3 significantly improve memory impairment in the 5*FAD mice:

FIG. 5 shows that OLB-3 significantly reduce the number of rotations in APO-induced 6-OHDA Parkinson's disease rats;

FIG. 6 shows effects of OLB-3 on a pole climbing time of ALS transgenic mice; and

FIG. 7 shows effects of OLB-3 on a limb grip force of the ALS transgenic mice.

### DETAILED DESCRIPTION OF THE EMBODIMENTS

### Example 1

### Synthesis of Compound OLB-3

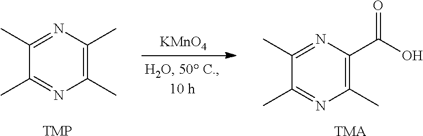

Tetramethylpyrazine (TMP) (13.6 g, 100.0 mmol) was dissolved in water (300 mL), potassium permanganate (31.6 g, 200.0 mmol) was added in portions, and a mixture was stirred at 50° C. for 10 h. After the reaction, a resulting product was cooled, adjusted to a pH value of 3 with hydrochloric acid, extracted with ethyl acetate, dried over anhydrous sodium sulfate, filtered and concentrated to obtain a product TMA (10.3 g, 62%). ¹H NMR (400 MHz, CDCl₃) δ 2.90 (s, 3H), 2.61 s, 3H), 2.56 s, 3H). MS ESI) m/z: 167.0 [M+H]⁺.

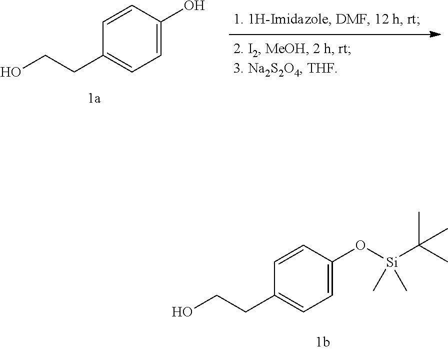 [SMILES: OCCc1ccc(O)cc1 ; CC(C)(C)[Si](C)(C)Oc1ccc(CCO)cc1]

Compounds imidazole (6.2 g, 90.5 mmol) and tert-butyldimethylsilyl chloride (13.6 g, 90.5 mmol) were dissolved in N,N-dimethylformamide (200 ml), a compound 1a (5.0 g, 36.2 mmol) was added in portions, and stirred at room temperature for a reaction overnight. After the reaction, a resulting product was diluted with water, extracted with n-hexane, dried over anhydrous sodium sulfate, filtered and concentrated; a part (3.7 g) of an obtained crude product was dissolved in methanol (40 mL), and elemental iodine (0.4 g) was added and stirred for 2 h; after the reaction, sodium thiosulfate was added for quenching, a resulting product was concentrated, diluted with ether, washed with water and then saturated brine, dried over anhydrous sodium sulfate, filtered, concentrated, and a product 1b (2.0 g, 83%) was obtained by silica gel column chromatography. ¹H NMR (400 MHz, DMSO-d₆) δ 6.92 (d, J=8.5 Hz, 2H), 6.69-6.49 (m, 2H), 4.41 (t, J=5.2 Hz, OH), 3.40 (td, J=7.1, 5.3 Hz, 2H), 2.49 (t, J=7.1 Hz, 2H), 0.78 (s, 9H), 0.07 (s, 6H). MS (ESI) m/z: 253.2 [M+H]⁺.

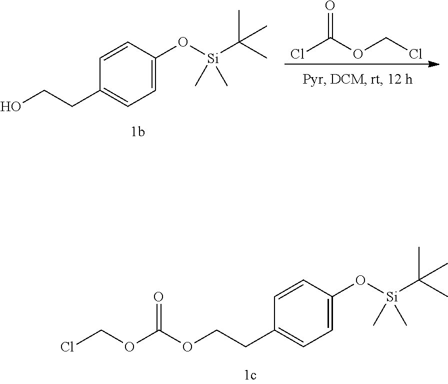

The compound 1b (830 mg, 3.3 mmol) and chloromethyl chloroformate (460 mg, 3.6 mmol) were dissolved in dichloromethane (20 ml), pyridine (0.3 mL) was added dropwise in an ice bath, and a mixture was stirred at room temperature overnight. After the reaction, a filtrate was collected by filtration and concentrated, and a product 1c was obtained by silica gel column chromatography (703 mg, 62%). ¹H NMR (400 MHz, CDCl₃) δ 6.88 (d, J=8.4 Hz, 2H), 6.59 (d, J=8.4 Hz, 2H), 5.52 (s, 2H), 4.19 (t, J=7.2 Hz, 2H), 2.75 (t, J=7.2 Hz, 2H), 0.79 (s, 9H), 0.00 (s, 6H). MS (ESI) m/z: 345.1 [M+H]⁺.

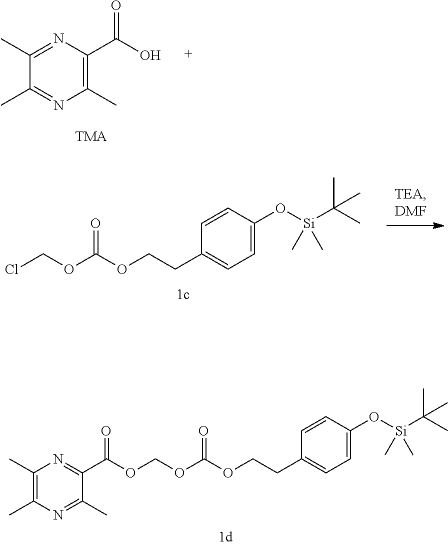

The compound TMA (332 mg, 2.0 mmol) and the compound 1c (688 mg, 2.0 mmol) were dissolved in N,N-dimethylformamide (20 mL), and stirred at 65° C. for 2 h. After the reaction, a resulting product was cooled, the reaction system was diluted by ethyl acetate, washed with water and then saturated brine in sequence, an organic phase was dried over anhydrous sodium sulfate, filtered, concentrated, and a product 1d (742 mg, 78%) was obtained by silica gel column chromatography. ¹H NMR (400 MHz, CDCl₃) δ 6.88 (d, J=8.4 Hz, 2H), 6.67-6.51 (m, 2H), 5.86 (s, 2H), 4.17 (t, J=7.2 Hz, 2H), 2.74 (t, J=7.2 Hz, 2H), 2.59 (s, 3H), 2.40 (s, 6H), 0.80 (s, 9H), 0.00 (s, 6H). MS (ESI) m/z: 475.2 [M+H]⁺.

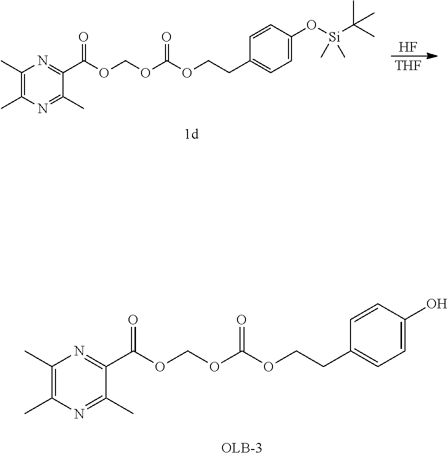

The compound 1d (95 mg, 0.2 mmol) was dissolved in tetrahydrofuran (10 mL), a hydrofluoric acid solution (1.0 ml, 2.0 mmol) was added, and a reaction was conducted by reflux for 1 h. After the reaction, a resulting product was washed with a saturated sodium bicarbonate solution, water and saturated brine in sequence, the organic phase was dried with anhydrous sodium sulfate, filtered, concentrated, and a product OLB-3 (60 mg, 84%) was obtained by silica gel column chromatography. ¹H NMR (400 MHz, CDCl₃) δ 7.05 (d, J=8.2 Hz, 2H), 6.74 (d, J=8.4 Hz, 2H), 6.02 (s, 2H), 4.32 (t, J=7.1 Hz, 2H), 2.90 (t, J=7.1 Hz, 2H), 2.76 (s, 3H), 2.58 (s, 3H), 2.57 (s, 3H). MS (ESI) m/z: 361.1 [M+H]⁺.

### Example 2 OLB-3 Significantly Reducing SH-SY5Y Cell Death Caused by OGD

A neuroprotective effect of tetramethylpyrazine (TMP) and derivatives thereof was evaluated by MTT assay. Cells were incubated, cells in the logarithmic growth phase were collected, a concentration of a cell suspension was adjusted, an MTT-containing medium was added after dosing treatment and 4 h of incubation with OGD, incubation was conducted for 4 h, the medium in the wells was carefully removed, and 150 μl of dimethyl sulfoxide (DMSO) was added to each well, followed by shaking at a low speed for 10 min on a shaker to fully dissolve crystals, and an absorbance value of each well was measured at an OD (absorbance) value of 490 nm of an enzyme-linked immunosorbent assay instrument (at the same time, a zero adjustment well (medium, MTT, DMSO) and a control well (cells, a drug dissolution medium with the same concentration, medium, MTT, DMSO) were set). Data were presented as mean±SEM; n=8 per group. One-way ANOVA and multiple comparisons showed differences between the two groups, a, p<0.001 vs. control group; b, p<0.05 vs OGD group; and c, p<0.001 vs. OGD group.

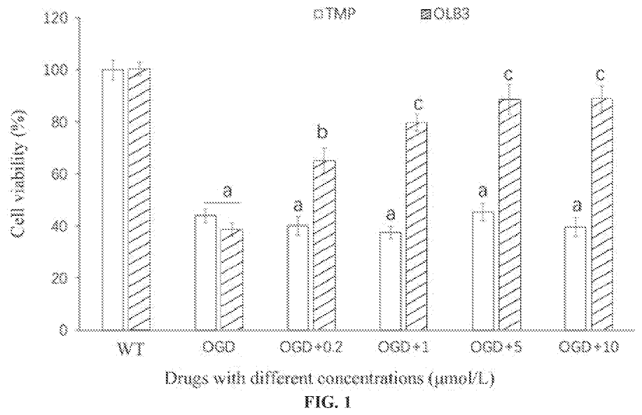

It can be seen from FIG. 1 that OLB-3 can significantly reduce the death of SH-SY5Y cells caused by OGD, and have neuroprotective effects.

### Example 3 OLB-3 Significantly Reducing Elevation of Inflammatory Factors and Oxidative Stress Caused by LPS

The SH-SY5Y cells were recovered and incubated, and the cells in the logarithmic growth phase were taken. After 24 h of incubation, SH-SY5Y neuroblastoma cells were treated with 1 μM all-trans retinoic acid to induce differentiation, and then inoculated into a 6-well culture dish for 24 h of incubation. Then, 0.2 μM (L) or 1 μM (H) OLB-3 and 1 μg/mL LPS were added to the medium, and treated for 24 h, a supernatant medium was aspirated, and changes of inflammatory factors and oxidative stress-related proteins were measured by an ELISA kit. Data were presented as mean±SEM; n=8 per group. One-way ANOVA and multiple comparisons showed differences between the two groups, a, p<0.05 vs. LPS group; b, p<0.01 vs LPS group; and c, p<0.001 vs. LPS group.

TABLE 1

| (pg/ml) | WT | LPS | LPS + TMP(L) | LPS + TMP(H) | LPS + OLB03(L) | LPS + OLB03(H) |
|---|---|---|---|---|---|---|
| TNF-alpha | 0.00 | 12.85 ± 1.45 | 12.22 ± 1.28 | 12.64 ± 1.70 | 7.34 ± 1.38(c) | 4.19 ± 0.77(c) |
| IL1 | 0.00 | 13.59 ± 1.63 | 13.26 ± 1.31 | 13.53 ± 1.74 | 8.76 ± 1.56(c) | 4.27 ± 0.58(c) |
| IL6 | 0.00 | 13.92 ± 1.13 | 14.12 ± 1.46 | 12.69 ± 1.85 | 9.34 ± 1.67(b) | 5.13 ± 0.63(c) |

TABLE 2

| | WT | LPS | LPS + TMP(L) | LPS + TMP(H) | LPS + OLB03(L) | LPS + OLB03(H) |
|---|---|---|---|---|---|---|
| SOD | 20.49 ± 1.09 | 9.40 ± 0.77 | 9.57 ± 0.61 | 8.93 ± 0.47 | 14.96 ± 1.32(c) | 18.27 ± 1.02(c) |
| (nU/ml) | | | | | | |
| MDA | 0.35 ± 0.02 | 23.75 ± 1.29 | 23.34 ± 1.71 | 22.94 ± 1.33 | 11.10 ± 0.78(c) | 6.71 ± 0.51(c) |
| (nmol/mg) | | | | | | |
| GSH-Px | 20.59 ± 1.05 | 5.90 ± 0.58 | 5.96 ± 0.74 | 6.90 ± 0.87 | 13.21 ± 1.92(c) | 17.51 ± 1.17(c) |
| (umol/mg) | | | | | | |

From Table and Table 2, it can be seen that OLB-3 significantly reduced the elevation of inflammatory factors and oxidative stress caused by LPS, and had strong anti-inflammatory and antioxidant effects.

### Example 4 OLB-3 Significantly Improving Abnormal Glucose and Lipid Metabolism in db/db Mice

Mice of a normal control group (WT) and model mice were given normal saline 10 ml/kg/d, Losartan 15 mg/kg/d, TMP (5.0 mg/kg, 0.037 mmol/kg), and OLB-3 (13.32 mg/kg, 0.037 mmol/kg), at a volume of 10 mL/kg, once/d, for continuous administration of 56 d, blood lipids and blood glucose related indexes were measured after blood collection. Data were presented as mean±SEM; n=6 per group. One-way ANOVA and multiple comparisons showed differences between the two groups. a, p<0.05 vs. db/db group; c, p<0.001 vs. db/db group.

TABLE 3

| | Glycated hemoglobin | Glucose determination | Total cholesterol | Triglyceride |
|---|---|---|---|---|
| | (nmol/L) | (mM) | (mM) | (mg/dL) |
| WT | 184.47 ± 10.16 | 7.75 ± 1.55 | 2.42 ± 0.11 | 9.11 ± 1.28 |
| db/db | 983.37 ± 41.26 | 39.71 ± 3.03 | 12.4 ± 1.05 | 13.81 ± 1.19 |
| db/db + metformin | 628.52 ± 50.59(c) | 24.01 ± 2.38(c) | / | / |
| db/db + Losartan | / | / | 6.76 ± 0.46(c) | 9.68 ± 0.37(c) |
| db/db + TMP | 917.31 ± 57.43 | 37.94 ± 3.11 | 10.96 ± 1.38 | 12.83 ± 1.37 |
| db/db + OLB3 | 517.83 ± 47.26(c) | 17.26 ± 1.42(c) | 4.15 ± 0.18(c) | 9.61 ± 0.57(c) |

| | High-density lipoprotein | Low-density lipoprotein | Urea | Creatinine |
|---|---|---|---|---|
| | cholesterol (mM) | cholesterol (mM) | (mmol/L) | (μmol/L) |
| WT | 1.45 ± 0.13 | 0.44 ± 0.03 | 6.86 ± 0.25 | 38.25 ± 1.33 |
| db/db | 2.365 ± 0.16 | 0.71 ± 0.03 | 12.06 ± 0.81 | 54.62 ± 3.34 |
| db/db + metformin | 2.19 ± 0.12(a) | 0.59 ± 0.02(c) | / | / |
| db/db + Losartan | / | / | 7.23 ± 0.51(c) | 42.12 ± 1.72(c) |
| db/db + TMP | 2.49 ± 0.16 | 0.68 ± 0.04 | 11.55 ± 0.59 | 52.5 ± 3.76 |
| db/db + OLB3 | 1.62 ± 0.11(c) | 0.52 ± 0.02(c) | 7.42 ± 0.31(c) | 42.42 ± 2.73(c) |

As can be seen from Table 3, OLB-3 significantly improved abnormal glucose and lipid metabolism, decreased the contents of total cholesterol and triglyceride, decreased the contents of high-density lipoprotein cholesterol and low-density lipoprotein cholesterol, and decreased the contents of urea and creatinine.

### Example 5 OLB-3 Significantly Improving Biochemical and Metabolic Indicators in db/db Mice

Mice of a normal control group (WT) and model mice were given normal saline 10 ml/kg/d, Losartan 15 mg/kg/d, TMP (5.0 mg/kg, 0.037 mmol/kg), and OLB-3 (13.32 mg/kg, 0.037 mmol/kg), at a volume of 10 mL/kg, once/d, for continuous administration of 56 d, blood lipids and blood glucose related indexes were measured after blood collection. Data were presented as mean f SEM; n=6 per group. One-way ANOVA and multiple comparisons showed differences between the two groups. a, p<0.05 vs. db/db group; c, p<0.001 vs. db/db group.

TABLE 4

| | Urine albumin/ | | |
|---|---|---|---|
| | creatinine | Urea | Creatinine |
| | (mg/g) | (mmol/L) | (μmol/L) |
| WT | 53.22 ± 15.76 | 6.86 ± 0.25 | 38.25 ± 1.33 |
| db/db | 792.37 ± 103.65 | 12.06 ± 0.81 | 54.62 ± 3.34 |
| db/db + | 582.74 ± 97.82(c) | 7.23 ± 0.51(c) | 42.12 ± 1.72(c) |
| Losartan | | | |
| db/db + TMP | 661.82 ± 89.54 | 11.55 ± 0.59 | 52.5 ± 3.76 |
| db/db + OLB3 | 443.82 ± 78.34(c) | 7.42 ± 0.31(c) | 42.42 ± 2.73(c) |

As can be seen from Table 4, OLB-3 significantly improved the biochemical and metabolic indicators of db/db mice, and decreased the contents of urea and creatinine.

### Example 6 OLB-3 Significantly Reducing Urinary Protein Levels in db/db Mice

Mice of a normal control group (WT) and model mice were given normal saline 10 ml/kg/d, Losartan 10 mg/kg/d, TMP (5.0 mg/kg, 0.037 mmol/kg), and OLB-3 (13.32 mg/kg, 0.037 mmol/kg), at a volume of 10 m/kg, once/d, for continuous administration of 56 d, urine protein levels were measured after urine collection. Data were presented as mean±SEM; n=6 per group. One-way ANOVA and multiple comparisons showed differences between the two groups. **, p<0.01 vs db/db group.

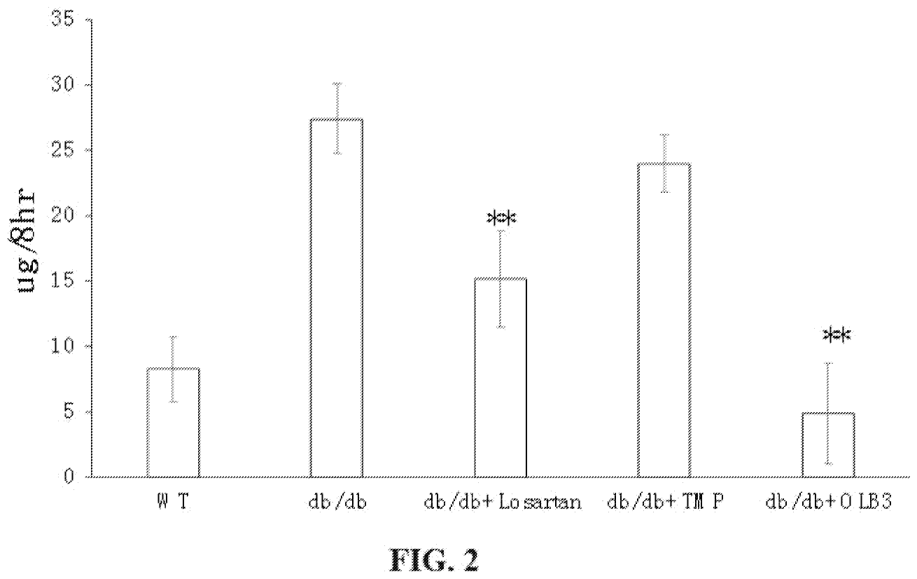

As shown in FIG. 2, OLB-3 significantly reduced urinary protein levels.

### Example 7 Use of OLB-3 in Treating ND

OLB-3 significantly improving memory impairment in 5*FAD mice

6-month-old 5*FAD mice were treated with OLB-3 for 3 months, and novel object recognition and Y-maze behavior were measured. Novel object recognition: during a training phase, the mice were exposed in a chamber to become familiar with two identical objects (A+A) for 5 min; during a testing phase, one of the familiar objects was replaced with another novel object (A+B), and the mice were put back in the chamber to detect probe objects for 5 min, while recording video and tracking the mice in real time. Probing was defined as a mouse with its nose pointed towards the object, sniffing or touching with its nose, and a distance of ≤2 cm was recorded from the nose to the object. A discriminant index (DI) is used to explore each object, and a calculation method thereof was as follows: (exploration time for new object−exploration time for old object)/(exploration time for new object+exploration time for old object)*100. Y-maze: an animal was placed at an end of one arm, and the sequence of the animals entering into each arm was recorded within 10 min. For the novel object recognition (A) test, it was found that a time ratio of the 5*FAD mice in OLB-3 treatment group to explore novel objects was significantly improved; for the Y-maze test (B) test, it was found that a time ratio of the 5*FAD mice the OLB-3 treatment group was significantly increased in a novel different arm. The 5*FAD mice were treated with low dosage (2.63 mg/kg, 0.007 mmol/kg, the same below) and high dosage (13.32 mg/kg, 0.037 mmol/kg, the same below) of the OLB-3 separately. Data were presented as mean±SEM; n=9-10 per group. One-way ANOVA and multiple comparisons showed differences between the two groups. **p<0.01, ***p<0.001 vs. WT (normal control) group; #p<0.05, ##p<0.01 vs. 5* FAD group.

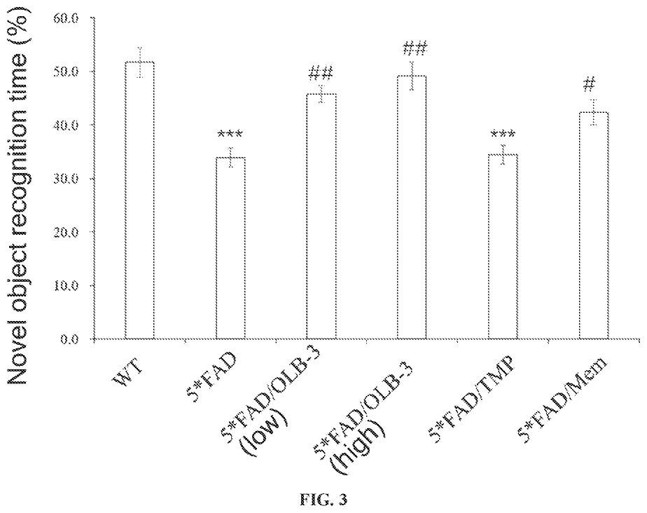

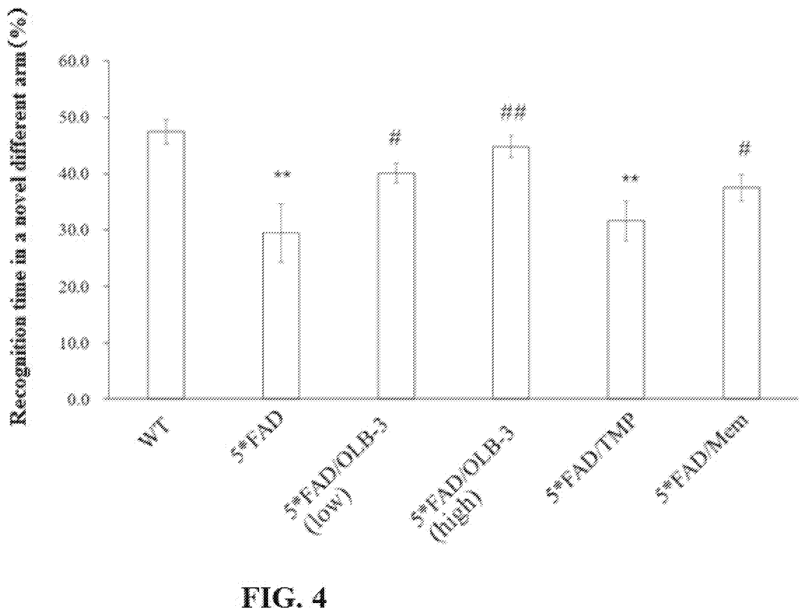

As can be seen from FIG. 3 and FIG. 4, OLB-3 can significantly increase a time ratio to explore novel objects, significantly increase a time ratio in a novel different arm, and improve the memory impairment.

OLB-3 significantly reducing the number of rotations in APO-induced 6-OHDA Parkinson's disease rats

After 3 weeks of modeling, the rotations of rats were recorded, the rats were induced to rotate, and behavioral changes of the rats were observed in a quiet and spacious environment. There was no rotations in a sham-operated group, and there was no significant difference between the groups injected with 6-OHDA, with about 180 rotations. After 2 weeks of treatment, the number of rotations increased slightly in the model group treated with normal saline; after 2 weeks of treatment with different dosages of OLB-3, TMP and positive control drug L-dopa, the results were as follows: the treatment with different dosages of the OLB-3 and positive control levodopa (25 mg/kg) can effectively reduce the number of rotations in APO-induced 6-OHDA rats. Compared with the 6-OHDA model group, OLB-3 treated rats showed a significant reduction in the number of rotations. Data were presented as mean±SEM; n=10 per group. One-way ANOVA and multiple comparisons showed differences between the two groups, *p<0.05, **p<0.01 vs. before 6-OHDA group.

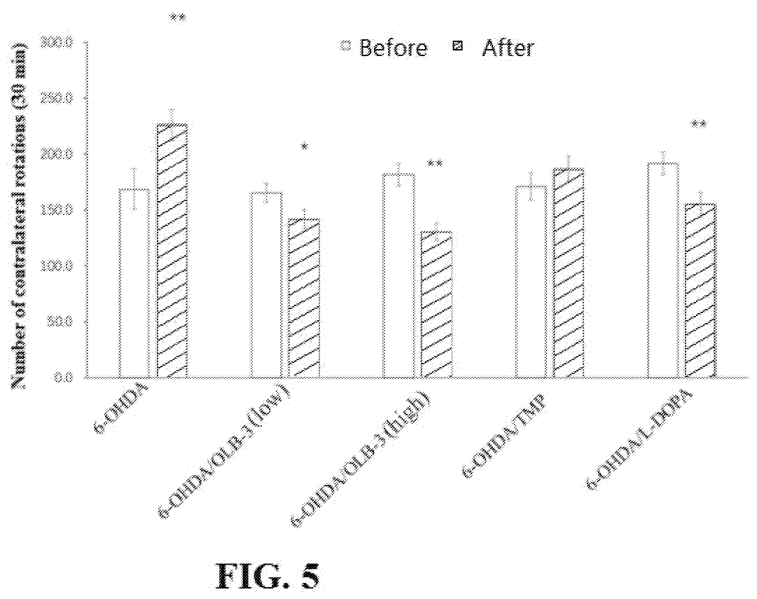

As shown in FIG. 5, OLB-3 had therapeutic effects on Parkinson's disease, significantly reducing the number of rotations.

Effects of OLB-3 on a Pole Climbing Time of ALS Transgenic Mice

The pole climbing test is generally used to evaluate a movement coordination ability, movement delay of the limbs in mice, and muscle strength. A homemade wooden pole about 50 cm long and about 1 cm in diameter was wrapped with medical gauze to increase friction of the wooden pole. The wooden pole was put vertically on a horizontal table, the mouse tail was grabbed such that the mouse head was facing down, with limbs grabbing a top of the pole; the timer was started after releasing the mouse tail, and it was ensured that the mouse crawled downward without external force, and the time was recorded for the mouse to climb from the top of the pole to a bottom platform (a standard was that mouse hind limbs were landed on the ground). Mice were continuously trained on this behavior for 3 d before administration, each mouse was repeated three times, and the mice that did not meet the standard were excluded. After the start of administration, the mouse behavior was tested every two weeks, a maximum of test results did not exceed 15 sec, and values exceeding 15 sec were recorded as 15 sec. The average value of the mouse's three pole climbing times was calculated as the final pole climbing time. ALS (SOD-G93A) mice developed obvious bradykinesia after the onset of disease, manifested as pole climbing time was significantly longer than that of control mice, and the bradykinesia becomed more severe with age. After treatment with different dosages of OLB-3, TMP and riluzole, it was found that OLB-3 and the positive control drug riluzole (5 mg/kg) both could significantly improve the symptoms of bradykinesia. Data were presented as mean±SEM; n=10 per group. One-way ANOVA and multiple comparisons showed differences between the two groups. *p<0.05 vs. WT (normal control) group; #p<0.05 vs. ALS (SOD-G93A) group.

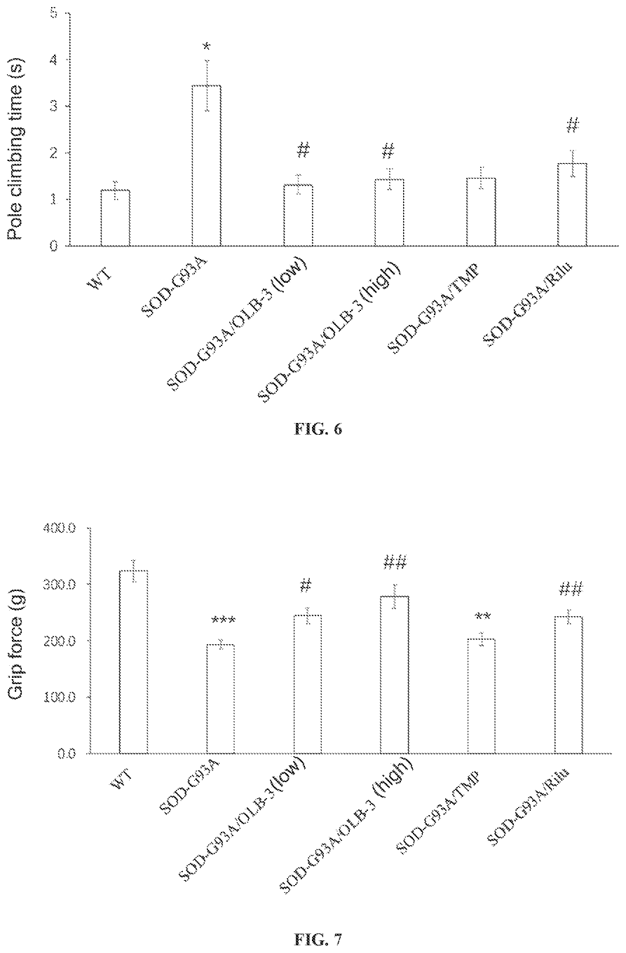

As shown in FIG. 6, OLB-3 had therapeutic effects on ALS, significantly shortening pole climbing time and improving bradykinesia.

Effects of OLB-3 on a limb grip force of ALS transgenic mice

The limb grip force test is used directly to assess muscle strength of mice and the incidence of mice. The mouse were placed on a central stage of a grip board, the mouse tail was gently pulled to urge the mouse to grasp the grip board, and the mouse was pulled backward and horizontally when the mouse firmly grasped a grip net, and the data were recorded once the instrument showed a maximum grip force. After the start of administration, the grip force of the mice was tested every two weeks, and the measurement was repeated three times for each mouse, and the maximum value among the three results was taken as a maximum grip force of the mice. After the ALS transgenic mice enter the disease stage, the limb grip force is significantly smaller than that of the WT mice. After treatment with different dosages of OLB-3, TMP and riluzole, it was found that OLB-3 and the positive control drug riluzole (5 mg/kg) both could effectively increase the limb force of mice, and delayed the deterioration of limb grip force decline in ALS mice. Data were presented as mean±SEM; n=10 per group. One-way ANOVA and multiple comparisons showed differences between the two groups, **p<0.01, ***p<0.001 vs. WT (normal control) group; #p<0.05, ##p<0.01 vs. ALS (SOD-G93A) group.

As shown in FIG. 7, OLB-3 had therapeutic effects on ALS, significantly improving limb grip force and enhancing muscle strength.

The above description of examples is only provided to help understand the method of the present disclosure and a core idea thereof. It should be noted that several improvements and modifications may be made by those skilled in the art without departing from the principle of the present disclosure, and these improvements and modifications should also fall within the protection scope of the present disclosure. Various amendments to these examples are obvious to those skilled in the art, and the general principles defined herein may be implemented in other examples without departing from the spirit or scope of the present disclosure. Thus, the present disclosure is not limited to the examples shown herein but falls within the widest scope consistent with the principles and novel features disclosed herein.

1. A pyrazine compound, a stereoisomer, and a tautomer, and a pharmaceutically acceptable salt thereof, wherein the pyrazine compound is shown in formula I: 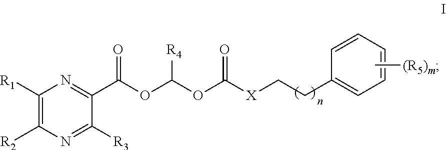
in formula I, wherein, X is selected from the group consisting of O, S, Se, and NR₆; R₁, R₂, R₃, R₄, R₅, and R₆ each are independently selected from the group consisting of H, deuterium, halogen, hydroxyl, amino, carboxyl, acylamino, ester, alkyl, alkenyl, alkynyl, cycloalkyl, heterocyclyl, alkoxy, alkylcarboxyl, alkylester, -alkyl-OH, alkoxy, alkylamino, -alkyl-NH₂, -aryl, heteroaryl, carbonate, carbamate, -alkyl-acylamino, -aminocarboxylate, and deuterated derivatives of the above groups; and n is 0 to 6, m is 0 to 5.

2. The pyrazine compound, the stereoisomer, and the tautomer, and the pharmaceutically acceptable salt thereof according to claim 1, wherein R₁, R₂, R₃, R₄, R₅, and R₆ each are independently selected from the group consisting of H, deuterium, halogen, hydroxyl, amino, carboxyl, acylamino, ester, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkoxy, substituted or unsubstituted alkylcarboxyl, substituted or unsubstituted alkylester, substituted or unsubstituted -alkyl-OH, substituted or unsubstituted alkoxy, substituted or unsubstituted alkylamino, substituted or unsubstituted -alkyl-NH₂, substituted or unsubstituted aryl, substituted or unsubstituted heterocyclic aryl, substituted or unsubstituted carbonate, substituted or unsubstituted carbamate, substituted or unsubstituted -alkyl-acylamino, substituted or unsubstituted -aminoalkylcarboxylate, and deuterated derivatives of the above groups.

3. The pyrazine compound, the stereoisomer, and the tautomer, and the pharmaceutically acceptable salt thereof according to claim 1, wherein n is 0, 1, 2, 3, 4, 5, or 6; and m is 0, 1, 2, 3, 4, or 5.

4. The pyrazine compound, the stereoisomer, and the tautomer, and the pharmaceutically acceptable salt thereof according to claim 1, wherein R₁, R₂, and R₃ each are selected from the group consisting of methyl and deuterated methyl, X is selected from the group consisting of O, S, Se, and NR₆; and R₄ is selected from the group consisting of H and C₁-C₆ alkyl.

5. The pyrazine compound, the stereoisomer, and the tautomer, and the pharmaceutically acceptable salt thereof according to claim 1, wherein n is 1, X is selected from the group consisting of O, S, Se, and NH; and R₄ is selected from the group consisting of H and C₁-C₆ alkyl.

6. The pyrazine compound, the stereoisomer, and the tautomer, and the pharmaceutically acceptable salt thereof according to claim 1, wherein X is O, n is 1; and R₄ is selected from the group consisting of H and C₁-C₆ alkyl.

7. The pyrazine compound, the stereoisomer, and the tautomer, and the pharmaceutically acceptable salt thereof according to claim 1, wherein the compound is shown as follows:

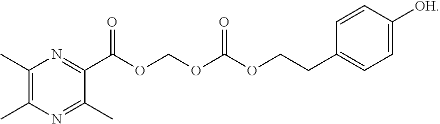 [SMILES: Cc1nc(C)c(C(=O)OCOC(=O)OCCc2ccc(O)cc2)nc1C]

8. The pyrazine compound, the stereoisomer, and the tautomer, and the pharmaceutically acceptable salt thereof according to any one of claim 1, wherein the pharmaceutically acceptable salt is a salt obtained by reaction of the pyrazine compound with hydrochloric acid, sulfuric acid, phosphoric acid, hydrobromic acid, nitric acid, salicylic acid, oxalic acid, benzoic acid, maleic acid, fumaric acid, citric acid, succinic acid, tartaric acid, C₁₋₆ fatty carboxylic acid, C₁₋₆ alkyl sulfonic acid, benzenesulfonic acid, p-toluenesulfonic acid, or camphorsulfonic acid.

9. A preparation method of a compound, comprising the following steps:

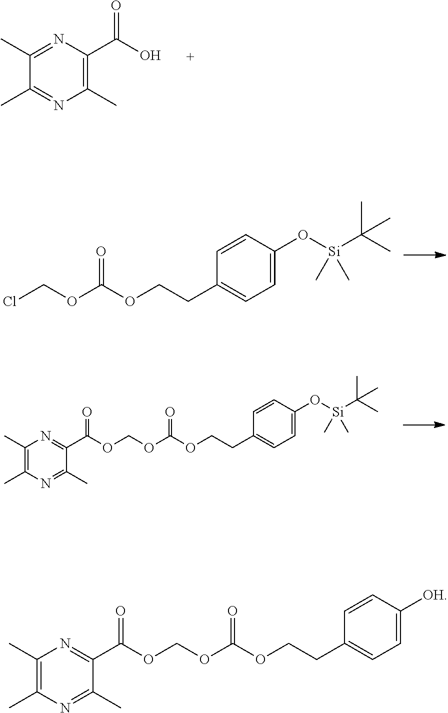

10. A compound, having a structural formula as follows:

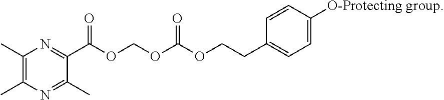

11. A compound, having a structural formula as follows:

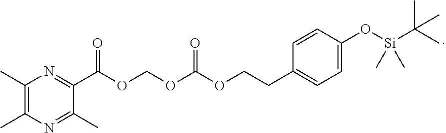 [SMILES: Cc1nc(C)c(C(=O)OCOC(=O)OCCc2ccc(O[Si](C)(C)C(C)(C)C)cc2)nc1C]

12. A method for treating a disease, comprising administering to a subject in need thereof the pyrazine compound, the stereoisomer, and the tautomer, and the pharmaceutically acceptable salt thereof according to claim 1, wherein the disease is selected from the group consisting of a neurodegenerative disease (ND), an inflammation, an oxidative damage, a mitochondrial disorder-related disease, diabetes mellitus (DM), and a DM-related complication.

13. A method for treating a disease, comprising administering to a subject in need thereof a compound according to claim 10, wherein the disease is selected from the group consisting of a ND, an inflammation, an oxidative damage, a mitochondrial disorder-related disease, DM, and a DM-related complication.

14. The method according to claim 12, wherein the ND comprises Alzheimer's disease, Parkinson's disease, Huntington's disease, frontotemporal dementia (FTD), vascular dementia, HIV-related dementia, multiple sclerosis, progressive lateral sclerosis, Friedreich's ataxia, neuropathic pain, and/or glaucoma.

15. A pharmaceutical composition, comprising a therapeutically effective amount of one or more of the pyrazine compound, the stereoisomer, and the tautomer, and the pharmaceutically acceptable salt thereof according to claim 1.

16. A pharmaceutical composition, comprising a therapeutically effective amount of the compound according to claim 10.

17. The pharmaceutical composition according to claim 15, further comprising one or more pharmaceutically acceptable carriers or excipients.

18. The pharmaceutical composition according to claim 15, wherein the pharmaceutical composition is capable of being prepared into a tablet, a granule, an injection, a gel, a pill, a capsule, a suppository, an implant, a nano preparation, or a powder for injection.

19. The method according to claim 1 2, wherein the administering is performed by oral, injection, subcutaneous, respiratory, transdermal, parenteral, rectal, topical, intravenous, intramuscular, or other means in a dosage unit formulation comprising a conventional pharmaceutically acceptable carrier.

20. The method according to claim 19, wherein the pharmaceutically acceptable carrier is selected from the group consisting of sugar, starch, cellulose, malt, gelatin, talc, and vegetable oil.

21. The method according to claim 13, wherein the compound has a structural formula as follows: 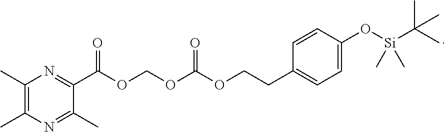 [SMILES: Cc1nc(C)c(C(=O)OCOC(=O)OCCc2ccc(O[Si](C)(C)C(C)(C)C)cc2)nc1C]
